(19) United States
(12) Patent Application Publication
(10) Pub. No.: US 2018/0326415 A1
(43) Pub. Date: Nov. 15, 2018

(54) MICROFLUIDIC DEVICE AND METHOD OF MANUFACTURE OF MICROFLUIDIC DEVICE

(71) Applicant: STRATEC CONSUMABLES GmbH, Anif (AT)

(72) Inventors: Dario Borovic, Hallein (AT); Thomas Umundum, Henndorf (AT)

(21) Appl. No.: 15/769,810

(22) PCT Filed: Feb. 12, 2016
(86) PCT No.: PCT/EP2016/052964

§ 371 Date: Apr. 20, 2018

(30) Foreign Application Priority Data
Mar. 5, 2015 (EP) 15157838.2

(51) Int. Cl. B01L 3/00; B29C 65/48; B29C 65/50; B29C 65/00; B81C 1/00; B29C 65/02; C12M 3/06

(52) U.S. Cl. CPC B01L 3/502707; B29L 2031/756; B29C 65/5057; B29C 66/026; B29C 66/112; B29C 66/1142; B29C 66/53461; B29C 66/5412; B29C 66/73118; B81C 1/00119; B29C 65/02; C12M 23/16; B01L 2300/0887; B01L 2300/163; B29C 66/71; B29C 65/4895

(57) ABSTRACT

A microfluidic device includes first and second outer layers each having one or more microfluidic formations and an intermediate layer bonded between the first and second outer layers; in which the glass transition temperature of the first outer layer is higher than the glass transition temperature of the second outer layer.

### CROSS REFERENCE TO RELATED APPLICATIONS

The present application claims the benefit of the earlier filing date of EP15157838.2 filed in the European Patent Office on 5 Mar. 2015, the entire contents of which application are incorporated herein by reference.

### BACKGROUND

### Field Of The Disclosure

This disclosure relates to microfluidic devices and methods of manufacture of microfluidic devices.

### Description of the Related Art

The “background” description provided herein is for the purpose of generally presenting the context of the disclosure. Work of the presently named inventors, to the extent it is described in this background section, as well as aspects of the description which may not otherwise qualify as prior art at the time of filing, are neither expressly or impliedly admitted as prior art against the present disclosure.

Microfluidic devices are used for fluid manipulation at a small scale, typically characterised by fluid volumes measured in μL (microliters). In a microfluidic device, fluids are manipulated within microfluidic channels or other formations, typically being formations provided in a structure of one or more layers by an etching, molding, laser cutting, milling, hot embossing or lithographic process.

### SUMMARY

This disclosure provides a microfluidic device including:

first and second outer layers each having one or more microfluidic formations; and

an intermediate layer bonded between the first and second outer layers;

in which the glass transition temperature of the first outer layer is higher than the glass transition temperature of the second outer layer.

Further respective aspects and features are defined in the appended claims.

It is to be understood that both the foregoing general description and the following detailed description are exemplary, but not restrictive of, the present disclosure.

### BRIEF DESCRIPTION OF THE DRAWINGS

A more complete appreciation of the disclosure and many of the attendant advantages thereof will be readily obtained as the same becomes better understood by reference to the following detailed description of embodiments, when considered in connection with the accompanying drawings, wherein:

FIGS. 1 to 3 are schematic cross-sections through respective example microfluidic devices;

FIG. 4 is a photograph of a cross-cut through an example microfluidic device illustrating a problem of so-called sagging;

FIGS. 5A and 5B schematically represent steps in a manufacture process; and

FIG. 6 is a photograph of a cross-cut through an example microfluidic device prepared using the steps of FIGS. 5A and 5B.

### DESCRIPTION OF THE EMBODIMENTS

The context of the present embodiments is as follows.

An example of a microfluidic device includes first and second outer (for example, microstructured) layers each having one or more microfluidic formations and a permeable or intermediate layer bonded between the first and second microstructured layers to allow material to permeate through the permeable layer from microfluidic formations in one of the first and second microstructured layers to microfluidic formations in the other of the first and second microstructured layers. Such an arrangement is useful in (for example) medical devices such as a so-called “organ on a chip” in which the activities, mechanics and/or physiological response of a human or animal organ system may be simulated. In such an example, material passage through the permeable layer from one to the other of the microstructured layers simulates at least a part of the operation of the organ system. As part of such a simulation, there may be biological cells, relating to the operation of the organ system, retained by the permeable layer.

The permeable layer may include (for example) a perforated film layer, bonded between two much more substantial microstructured layers.

A problem which can occur during manufacture of such a device is so-called “sagging”. This relates to a tendency of the permeable layer not to remain a flat, planar layer but, in regions corresponding to microstructured formations in one or both layers, to warp away from the plane of the permeable layer towards one or other of the microstructured formations.

At least example embodiments address the issue of sagging.

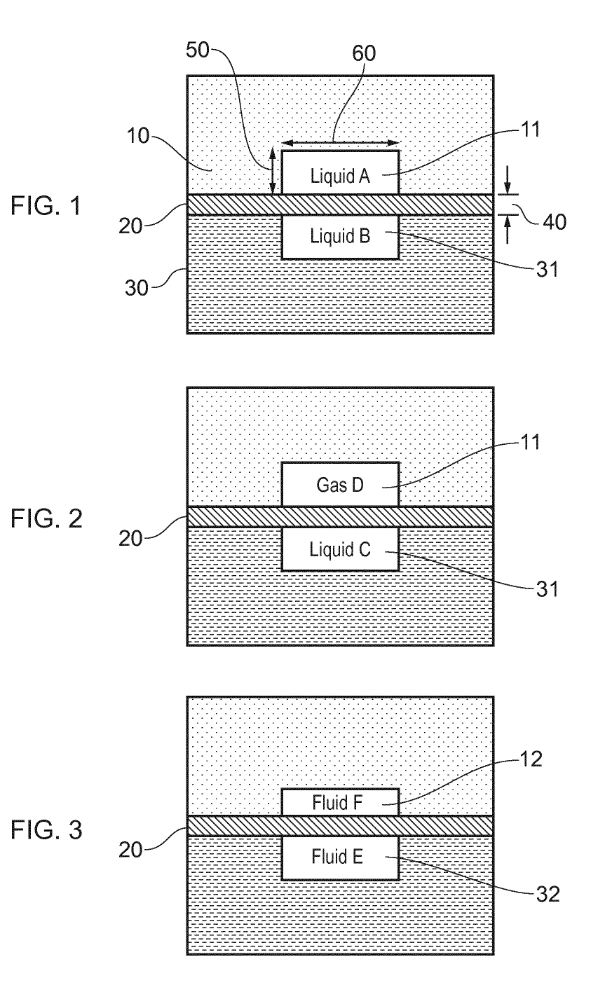

Referring now to the drawings, FIGS. 1 to 3 are schematic cross-sections through respective example microfluidic devices.

Each of the examples of FIGS. 1-3 relates to a so-called “organ on a chip” medical device, although the principles to be discussed are applicable to other types of microfluidic devices such as devices having microstructured formations either side of a permeable layer. In an example configuration, the complete unit may be substantially planar and of a size similar to that of a microscope slide. Microfluidic channels (shown in cross-section in FIGS. 1-3) are provided in the plane of the device and are provided with fluid inputs and outputs so that fluids may be introduced into and retrieved from the microfluidic channels.

Referring to FIG. 1, a microfluidic device includes a first microstructured or outer layer 10, an intermediate layer such as a permeable layer 20 and a second microstructured or outer layer 30 such that the permeable layer 20 is bonded between the first and second outer layers. Note that the word “outer” refers simply to the relationship amongst the three layers just described, in that the intermediate layer is between the two outer layers in the assembled layer structure. The word “outer” does not imply or provide any restriction on further layers or other features being provided at the outer periphery of the three-layer structure just described.

In particular, the permeable layer is bonded between the first and second microstructured layers to allow material to permeate through the permeable layer from microfluidic formations in one of the first and second microstructured layers to microfluidic formations in the other of the first and second microstructured layers.

In this context, the term “permeable” includes at least the sense that the permeable layer can be permeated or penetrated by fluids (liquids and/or gases) or by other material dissolved in or otherwise carried by such a fluid so that the fluids or material can pass through openings or interstices of the permeable layer, for example by a process of osmosis or diffusion. At a general level, this will be referred to as “material” permeating the permeable layer, where at a general level the term “material” encompasses fluids as well as dissolved or otherwise carried materials. An example of a permeable layer 20 is a film or membrane layer such as a so-called film (such as a thin film) layer, where the term “thin” refers to a layer thickness 40 of no more than 100 μm, though in some examples films of up to 300 μm could be used as the thin film. Suitable materials for the permeable layer 20 are discussed below. In an example embodiment, a film thickness of between 20 and 25 μm is used, with the film being formed of track-etched polycarbonate. However, track etched membranes or films are available in thicknesses from 5 to 100 μm, although laser processed membranes or membranes perforated by other perforation processes could also be used. For some applications a film thickness of 10-70 μm can be useful, on account of the cell culture requirements of some embodiments.

Therefore, an example range of thicknesses applicable to the definition of the “thin film” is 10-300 μm. Another example range of thicknesses applicable to the definition of the “thin film” is 10-100 μm. For so-called “cell culture” applications (in which cells or other biological material are grown, deposited or otherwise provided, on or in the film, as part of the intended operation of the system) an example range of thicknesses applicable to the definition of the “thin film” is 10-70 μm.

The permeable layer 20 is perforated so as to allow the fluids discussed above to permeate through the permeable layer 20. For example, a regular array of individual perforations, each approximately 3 μm across, and separated by 10-50 μm, can be provided in the permeable layer 20, for example by using a track etching, laser machining, hot embossing, spin coating, lithography or etching process.

Each of the microstructured layers 10, 30 includes one or more microfluidic formations such as microfluidic channels 11, 31. The microfluidic channels 11, 31 are arranged, at least in relation to portions where material exchange is desirable, to coincide with one another so that the microfluidic channel in one of the microstructured layers is aligned with that in the other microstructure layer with just the permeable layer forming a barrier between them. Example dimensions of the microfluidic channels are a channel depth 50 of (say) 0.05-1.5 mm and a channel width 60 of (say) 0.05-2 mm.

In the example shown in FIG. 1, the microfluidic device is arranged to receive a first liquid, liquid A, in the microfluidic channels 11 of the layer 10, and a second liquid, liquid B, in the microfluidic channels 31 of the layer 30. The choice of liquids depends upon the function being tested or simulated by the device. For example in the case of a device intended to simulate a part of the operation of the human kidney, one of the liquids may be blood and the other may be urine (or a precursor filtrate in the production of urine). Permeation of material through the permeable layer 40 in this example can simulate the operation of the nephron (a functional unit of the kidney) in its role of moving waste products from the blood into the urine. It will be understood that this simulation operation can be useful in at least two respects: in the testing of medicaments or other forms of treatment (in which it can be used to at least partially avoid the need for animal testing), and in the provision of artificial organ functions (such as in a dialysis process). In other examples, different organs can be simulated using similar techniques.

As part of the simulation process, for example in a “cell culture” application, it may be appropriate that the permeable layer retains on its surface or within its perforations biological cells, such as biological cells relating to the medical function being simulated. Such cells can be applied to the permeable layer before the device is first assembled, but there is the risk of contamination and of cell death or damage during the bonding process. In another option, therefore, such cells are introduced into the microfluidic device through at least a subset of the microfluidic channels, for example in an aqueous or other solution, and then (if appropriate) grown or propagated in place at the permeable layer (for example by providing appropriate nutrients, temperatures and time periods) before the simulation operation of the microfluidic device is started.

The example of FIG. 1 relates to a system in which material is exchanged between two liquids in the microfluidic channels 11, 31. In an alternative example shown in FIG. 2, in which all of the physical parts are the same as those shown in FIG. 1 (and so will not be described again) unless otherwise indicated, the microfluidic channel 31 carries a liquid, liquid C and the microfluidic channel 11 carries a gas, gas D. So, in contrast to the arrangement shown in FIG. 1, in which material dissolved or otherwise carried by one of liquids (for example blood) permeated through the permeable layer 20 into the other liquid (for example, a filtrate forming a precursor in the generation of urine), in the example of FIG. 2 one of the fluids itself can permeate through the permeable layer 20 into the other fluid. For example, the arrangement of FIG. 2 can be used in the simulation of a lung function. In an example of such an arrangement, biological cells are introduced to the permeable layer 20, for example human alveolar epithelial cells may be grown on one side of the permeable layer 20, while human pulmonary microvascular endothelial cells may be grown on the other side of the permeable layer 20. In an alternative example shown in FIG. 3, in which all of the physical parts are the same as those shown in FIG. 1 (and so will not be described again) unless otherwise indicated, the microfluidic channel 32, 12 have different depths. For example, the microfluidic channels 32 may be 1 mm deep whereas the microfluidic channels 12 may be 0.15 mm deep. Each of the channels carries a respective fluid, fluids E, F. Such an arrangement can be appropriate in the case of fluids of different viscosities and/or different concentrations of a relevant material and/or different desired flow rates.

A previously proposed assembly process for this type of device includes forming the three main parts or components of the device (first and second microstructured layers and the permeable layer) and executing a single thermal bonding (or solvent-assisted thermal bonding) process to bond the three components together to form the device. However, an issue which can arise during such a process is so-called sagging.

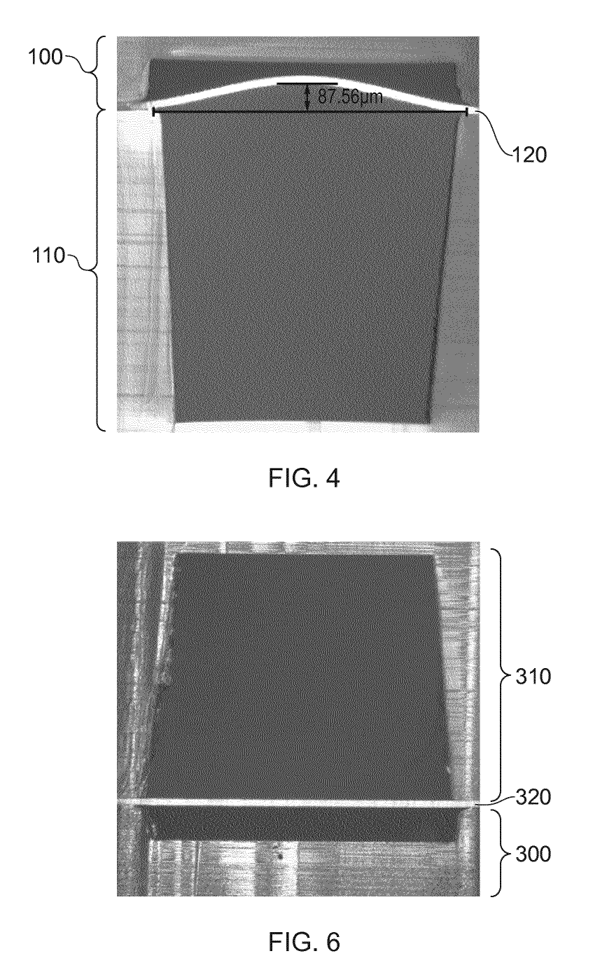

FIG. 4 is a photograph of a cross-cut through an example microfluidic device illustrating a problem of so-called sagging. The example device of which FIG. 4 is a photograph is similar in structure to that shown in schematic form in FIG. 3, and is formed of a pair of microstructured layers 100, 110 separated by a permeable layer 120. A microfluidic channel in the layer 100 is 0.15 mm deep, and a microfluidic channel in the layer 110 is 1 mm deep. The permeable layer 120 should be flat and horizontal (as represented in the orientation of the photograph) so as to form a planar boundary between the layers 100, 110. However, it can be seen that the permeable layer 120 has warped or bulged towards the layer 100 in this example (though in other examples the warping could be in the opposite sense). The furthest excursion from the desired plane of the permeable layer 120 has been measured from the photograph as 87.56 μm. Given that the channel depth in the layer 100 is only 150 μm (0.15 mm) the bulge of nearly 88μm causes a significant narrowing or impediment to flow along the channel in the layer 100. Also, in the case of a microscopic examination of cells on the permeable layer, sagging will mean that the focus will need to be adjusted between different regions of the permeable layer. Accordingly, this so-called sagging effect is undesirable.

Note that although the term “sagging” may suggest a gravitational warp or droop of the permeable layer 120, this is not in fact believed to be the mechanism by which the sagging occurs. Indeed, the sagging can take place in a direction which is unrelated to the orientation (with respect to gravity) of the device during manufacture or subsequent handling. In fact, the sagging is understood to occur because of material flow of the side walls of both microfluidic channels towards the channel center during the thermal or solvent-assisted thermal bonding process.

Note that a cross-cut examination is just one technique for detecting sagging. Other detection techniques include using a microscope from above or below the plane of the device, to view the permeable layer using a very shallow depth of field so that if sagging is present only some, but not all, of the permeable layer will be in focus, or to use an optical profiler, a type of interferometric arrangement.

Embodiments of the present disclosure address this issue by providing a two-stage bonding process. In a first bonding stage, the permeable layer is bonded to one of the microstructured layers. In a second, separate, bonding stage, the other of the microstructured layers is bonded to the other side of the permeable layer. In order to carry out such a two-stage process without the second bonding stage disturbing the bond already performed during the first stage, a material is used for the microstructured layer bonded during the first stage which has a higher glass transition temperature (Tg) than the glass transition temperature of the microstructured layer bonded during the second stage.

Example embodiments provide a microfluidic device including:

first and second outer layers each having one or more microfluidic formations; and

an intermediate layer bonded between the first and second outer layers;

in which the glass transition temperature of the first outer layer is higher than the glass transition temperature of the second outer layer.

A glass transition is a reversible transition in an amorphous material from a hard and relatively brittle state into a molten state. The transition is not in fact a phase transition but takes place around a characteristic temperature, the glass transition temperature (Tg). The definition of a glass transition temperature is by convention, because the transition occurs over a range of temperatures, but there are laboratory techniques and measurement conventions which lead to the derivation of a single value of Tg in respect of a particular amorphous material. For the present purposes, the particular measurement techniques and conventions used in the definition (which are of themselves known) are not relevant to the present discussion, except to say that in comparing the values of Tg between different materials the same conventions are used in the definitions of the respective values of Tg.

In terms of the bonding process is being discussed here the glass transition temperature is relevant to the bonding operation. In the examples to be discussed below, solvent-assisted thermal bonding is carried out. Here, a solvent is applied to a surface to be bonded (in this example, to the bonding surfaces of the microstructured layers) which has the effect of locally decreasing the glass transition temperature at the bonding surface. Once the solvent has taken effect, a thermal bonding process is applied so that the parts to be bonded are heated to a temperature approximating the glass transition temperature (as modified by the solvent). At the bonding temperature the material at the surface of the microstructured layer transitions to a molten state and bonding takes place with the permeable layer. The bonded arrangement is then cooled down to below the glass transition temperature.

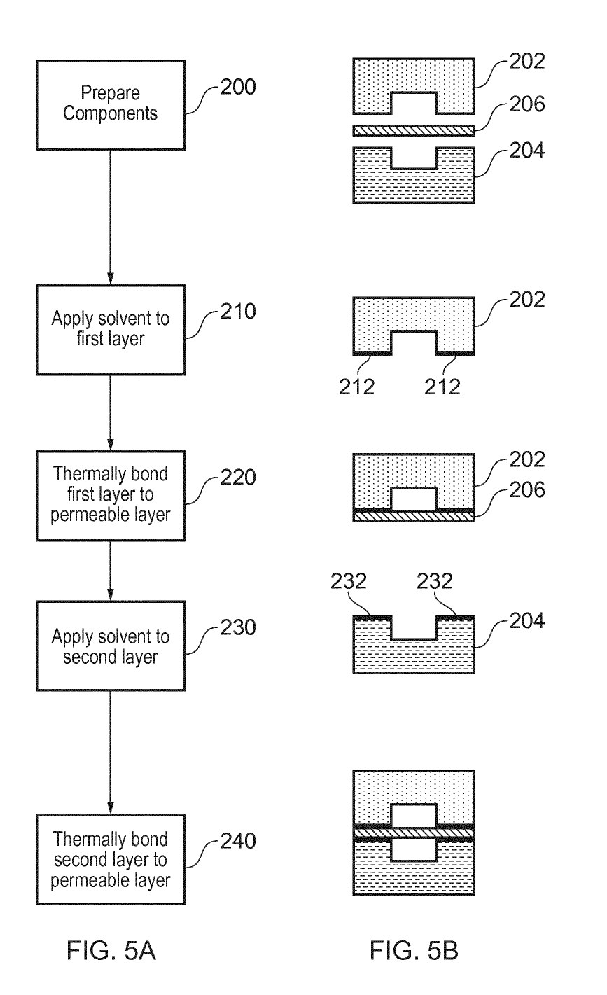

FIGS. 5A and 5B schematically represent steps in a manufacture process. In particular, FIG. 5A provides a schematic flowchart. FIG. 5B provides schematic illustrations, horizontally aligned with respective flowchart steps, to assist in and understanding of the bonding and manufacturing process. The combination of FIGS. 5A and 5B provides an example of a method of manufacture of a microfluidic device, the method including: bonding a first outer layer having one or more microfluidic formations and having a first glass transition temperature to an intermediate layer; and bonding a second outer layer having one or more microfluidic formations and having a second glass transition temperature to the intermediate layer, so as to form a microfluidic device; in which the first glass transition temperature of the first outer layer is higher than the second glass transition temperature of the second outer layer.

In some examples, the first bonding temperature is higher than the second bonding temperature. But in other examples this may not necessarily be the case. The first bonding step bonds a thin film (in this example) or other film or membrane to a substrate layer, which means that the bonding surface itself can be directly heated. But in the second bonding step, in which this two layer structure is bonded to another substrate layer, the bonding surfaces are not directly heated, and indeed can be considered to be insulated from the applied heat by the bulk of the substrate layers themselves. So in such examples, the second bonding temperature (in terms of a temperature set in respect of an oven or the like in which the bonding takes place) might actually be higher than that applied at the first step, but the effect of such temperature at the bonding surfaces does not necessarily reflect this relationship between the two bonding temperatures.

At a step 200, the respective components (first and second microstructured layers 202, 204 and a permeable layer 206) are prepared. As discussed above, the microstructured layers 202, 204 can be prepared from respective substrates by a molding, laser cutting, milling, hot embossing or lithographic process. The permeable layer 206 can be prepared from a film or membrane (such as a thin film) substrate by track etching, laser machining, hot embossing, spin coating, lithography or etching. The glass transition temperatures (Tg) differ between the two microstructured layers so that the layer which is bonded first has a higher Tg, for example at least 20° C. higher than that of the layer bonded second.

At a step 210, solvent is applied to a bonding surface 212 of one of the microstructured layers 202. As discussed above, the solvent has the effect of locally lowering the glass transition temperature of the material of that layer 202.

At a step 220, the solvent-treated layer 202 is thermally bonded to the permeable layer 206 at a first bonding temperature. This can be carried out in a press device so that the two parts are pressed together during the bonding process, and are then allowed to cool to well below the glass transition temperature.

At a step 230, solvent is applied to a bonding surface 232 of the other microstructured layer 204.

Finally, at a step 240, the solvent-treated layer 204 is aligned with the layer 202 and is thermally bonded to the permeable exposed side of the layer 206 at a second bonding temperature. Again, this can be carried out in the press device so that the two parts are pressed together during the bonding process, and are then allowed to cool to well below the glass transition temperature.

The glass transition temperature of the first of the layers (the layer 202 in the above example) to be bonded is higher than the glass transition temperature of the second of the layers (the layer 204 in the above example) to be bonded.

Examples of suitable materials for the outer layers, in any permutation subject to the constraint that the glass transition temperature (Tg) of the first outer layer (the layer to which the intermediate layer is first bonded) is higher than Tg of the second outer layer, include:
Polycarbonate (PC) (Tg=140° C.);
Cyclo-Olefin-Polymer (COP) (Tg=69° C.);
COP (Tg=100° C.);
COP (Tg=136° C.);
Polymethylmethacrylate PMMA (Tg=105° C.);
Polyethylenterephthalate (PET) (Tg=70° C.);
Cyclo-Olefin-Copolymer (COC) (Tg=78° C.);

1 COC(Tg=130° C.);
COC(Tg=150° C.); and
COC(Tg=170° C.).

Examples of suitable materials for the intermediate layer include:
Polycarbonate (Tg=140° C.);
Polyethylenterephthalate (PET) (Tg=70° C.);
COP/COC (Tg =70° C.-170° C.); and
PMMA (Tg=105° C.).

Examples of a selection of suitable materials for the first outer layer include:
Polycarbonate (PC) (Tg=140° C.);
Cyclo-Olefin-Polymer (COP) (Tg=100° C.);
COP (Tg=136° C.);
COP (Tg=163° C.);
Polymethylmethacrylate (PMMA) (Tg=105° C.);
COC (Tg=130° C.);
COC (Tg=150° C.); and
COC (Tg=170° C.).

Examples of a selection of suitable materials for the second outer layer include:
Polycarbonate (PC) (Tg=140° C.);
Cyclo-Olefin-Polymer (COP) (Tg=69° C.);
COP (Tg=100° C.);
Polymethylmethacrylate PMMA (Tg=105° C.);
Polyethylenterephthalate (PET) (Tg =70° C.);
Cyclo-Olefin-Copolymer (COC) (Tg=78° C.);and
COC (Tg=130° C.).

More generally, however, the outer layers and the intermediate layer can be provided as any permutation of COPs, COCs, PMMAs, PCs, Polystyrols, Polyethylenterephthalates (PETs), and Polyamides, subject to the constraint that the Tg of the first outer layer (the layer to which the intermediate layer is first bonded) is higher than the Tg of the second outer layer.

Example solvents include Chloroform, Trifluorethanol, Cyclohexane, Dichlormethane, Diacetonalcohol, Methylethylketone and Tetrafluorpropanol.

The techniques of FIGS. 5A and 5B can be used to manufacture a microfluidic device such as any of the devices illustrated schematically in FIGS. 1-3. The finished device is distinguished from previously proposed devices by at least the feature of the Tg values of the two microstructured layers. The manufacture technique is distinguished from previously proposed techniques by at least that feature and also the two-stage bonding process discussed above, using different bonding temperatures.

Such a device may be used in a medical device configured to simulate the activities, mechanics and/or physiological response of a human or animal organ system. The microfluidic device may be configured to receive a fluid into the microfluidic formations of at least one of the first and second microstructured layers, so that material passage through the permeable layer to the other of the first and second microstructured layers simulates at least a part of the operation of the organ system. Such a medical device may, as discussed above, include biological cells, relating to the operation of the organ system, retained by the permeable layer. FIG. 6 is a photograph of a cross-cut through an example microfluidic device prepared using the steps of FIGS. 5A and 5B. This photograph is shown in a different orientation to that of FIG. 4, in that a layer 300 having a shallow microfluidic channel depth (for example, 0.15 mm) is at the bottom of the photograph, whereas a layer 310 having a deeper channel (for example, 1 mm deep) is shown at the other part of the photograph. However, it will be appreciated that the orientation of the devices either during manufacture or in use is immaterial.

A permeable layer 320 has been bonded between the layers 300, 310. Inspection of the photograph of FIG. 6 shows that the permeable layer 320 has remained planar, which is to say that the so-called “sagging” issue discussed above has been avoided or at least substantially alleviated.

It will be apparent that numerous modifications and variations of the present disclosure are possible in light of the above teachings. It is therefore to be understood that within the scope of the appended claims, the technology may be practised otherwise than as specifically described herein.

Further respective aspects and features of the present disclosure are defined by the following numbered clauses:

1. A microfluidic device including:
first and second outer layers each having one or more microfluidic formations; and
an intermediate layer bonded between the first and second outer layers;
in which the glass transition temperature of the first outer layer is higher than the glass transition temperature of the second outer layer.

2. A microfluidic device according to clause 1, in which the intermediate layer is a permeable layer to allow material to permeate through the permeable layer from microfluidic formations in one of the first and second outer layers to microfluidic formations in the other of the first and second outer layers.

3. A microfluidic device according to clause 1 or clause 2, in which the outer layers and the intermediate layer are each formed of a respective material selected from the list consisting of:
Polycarbonates;
Cyclo-Olefin-Polymers;
Cyclo-Olefin-Copolymers;
Polymethylmethacrylates;
Polystyrols;
Polyethylenterephthalates (PETs); and
Polyamides

4. A microfluidic device according to clause 3, in which the intermediate layer is a film layer.

5. A microfluidic device according to clause 3 of clause 4, in which the intermediate layer is formed of a material selected from the list consisting of:
Polycarbonate (Tg=140° C.);
Polyethylenterephthalate (PET) (Tg=70° C.);
COP/COC (Tg=70° C.-170° C.); and
PMMA (Tg=105° C.).

6. A microfluidic device according to any one of clauses 3 to 5, in which the intermediate layer is perforated by using any one of a track etching, laser machining, hot embossing, spin coating, lithography or etching process.

7. A microfluidic device according to any one of clauses 3 to 6, in which the first outer layer is formed of a material selected from the list consisting of:
Polycarbonate (PC) (Tg=140° C.);
Cyclo-Olefin-Polymer (COP) (Tg=100° C.);
COP (Tg=136° C.);
COP (Tg=163° C.);
Polymethylmethacrylate (PMMA) (Tg=105° C.);
COC (Tg=130° C.);
COC (Tg=150° C.); and
COC (Tg=170° C.).

8. A microfluidic device according to any one of clauses 3 to 7, in which the second outer layer is formed of a material selected from the list consisting of:
Polycarbonate (PC) (Tg=140° C.);
Cyclo-Olefin-Polymer (COP) (Tg=69° C.);
COP (Tg=100° C.);
Polymethylmethacrylate PMMA (Tg=105° C.);
Polyethylenterephthalate (PET) (Tg=70° C.);
Cyclo-Olefin-Copolymer (COC) (Tg=78° C.);and
COC (Tg=130° C.).

9. A medical device configured to simulate the activities, mechanics and/or physiological response of a human or animal organ system, the medical device including a microfluidic device according to any one of the preceding clauses, the microfluidic device being configured to receive a fluid into the microfluidic formations of at least one of the first and second outer layers, so that material passage through the intermediate layer to the other of the first and second outer layers simulates at least a part of the operation of the organ system.

10. A medical device according to clause 9, including biological cells, relating to the operation of the organ system, retained by the intermediate layer.

11. A method of manufacture of a microfluidic device, the method including:
bonding a first outer layer having a first glass transition temperature to an intermediate layer; and
bonding a second outer layer having a second glass transition temperature to the intermediate layer;

in which the first glass transition temperature of the first outer layer is higher than the second glass transition temperature of the second outer layer. 12. A method according to clause 11, in which the bonding steps include applying a solvent to a surface to be bonded of the respective outer layer so as to locally reduce the glass transition temperature at the surface. 13. A method according to clause 11 or clause 12, in which the intermediate layer is a film layer. 14. A method according to any one of clauses 11 to 13, including perforating the intermediate layer by using a track etching, laser machining, hot embossing, spin coating, lithography or etching process.

1. A microfluidic device comprising:
first and second outer layers each having one or more microfluidic formations; and an
intermediate layer bonded between the first and second outer layers;
in which the glass transition temperature of the first outer layer is higher than the glass transition temperature of the second outer layer;
in which the intermediate layer is a permeable film layer to allow material to permeate through the permeable layer from microfluidic formations in one of the first and second outer layers to microfluidic formations in the other of the first and second outer layers.

2. canceled.

3. A microfluidic device according to claim 1, in which the outer layers and the intermediate layer are each formed of a respective material selected from the list consisting of:
Polycarbonates (PC);
Cyclo-Olefin-Polymers (COPs);
Cyclo-Olefin-Copolymers (COCs);
Polymethylmethacrylates (PMMAs);
Polystyrols;
Polyethylenterephthalates (PETs); and
Polyamides.

4. canceled.

5. A microfluidic device according to claim 1, in which the intermediate layer is formed of a material selected from the list consisting of:
Polycarbonate (Tg=140° C.);
Polyethylenterephthalate (PET) (Tg=70° C.);
COP/COC (Tg=70° C.-170° C.); and
PMMA (Tg=105° C.).

6. A microfluidic device according to claim 3, in which the intermediate layer is perforated by using any one of a track etching, laser machining, hot embossing, spin coating, lithography or etching process.

7. A microfluidic device according to claim 2, in which the first outer layer is formed of a material selected from the list consisting of:
Polycarbonate (PC) (Tg=140° C.);
Cyclo-Olefin-Polymer (COP) (Tg=100° C.);
COP (Tg=136° C.);
COP (Tg=163° C.);
Polymethylmethacrylate (PMMA) (Tg=105° C.);
COC (Tg=130° C.);
COC (Tg=150° C.); and
COC (Tg=170° C.).

8. A microfluidic device according to claim 2, in which the second outer layer is formed of a material selected from the list consisting of:
Polycarbonate (PC) (Tg=140° C.);
Cyclo-Olefin-Polymer (COP) (Tg=69° C.);
COP (Tg=100° C.);
Polymethylmethacrylate PMMA (Tg=105° C.);
Polyethylenterephthalate (PET) (Tg=70° C.);
Cyclo-Olefin-Copolymer (COC) (Tg=78° C.); and
COC (Tg=130° C.).

9. A medical device configured to simulate the activities, mechanics and/or physiological response of a human or animal organ system, the medical device comprising a microfluidic device according to claim 1 , the microfluidic device being configured to receive a fluid into the microfluidic formations of at least one of the first and second outer layers, so that material passage through the intermediate layer to the other of the first and second outer layers simulates at least a part of the operation of the organ system.

10. A medical device according to claim 7, comprising biological cells, relating to the operation of the organ system, retained by the intermediate layer.

11. A method of manufacture of a microfluidic device, the method comprising:
thermally bonding a first outer layer having a first glass transition temperature to an intermediate layer; and
thermally bonding a second outer layer having a second glass transition temperature to the intermediate layer so as to form a microfluidic device;
wherein the first glass transition temperature of the first outer layer is higher than the second glass transition temperature of the second outer layer; and
wherein the intermediate layer is a permeable film layer to allow material to permeate through the permeable layer from microfluidic formations in one of the first and second outer layers to microfluidic formations in the other of the first and second outer layers.

12. A method according to claim 9, in which the bonding steps comprise applying a solvent to a surface to be bonded of the respective outer layer so as to locally reduce the glass transition temperature at the surface.

13. canceled.

14. A method according to claim 9, comprising perforating the intermediate layer by using a track etching, laser machining, hot embossing, spin coating, lithography or etching process.
